(19) United States
(12) Patent Application Publication
(10) Pub. No.: US 2004/0058352 A1
(43) Pub. Date: Mar. 25, 2004

(54) Method of establishing resistance profiles of tissues and cell lines

(76) Inventors: Ulrike Stein, Schwanebeck (DE); Peter Michael Schlag, Berlin (DE)

Correspondence Address: BIRCH STEWART KOLASCH & BIRCH, PO BOX 747, FALLS CHURCH, VA, 22040-0747, US

(21) Appl. No.: 10/415,491

(22) PCT Filed: Sep. 3, 2001
(86) PCT No.: PCT/DE01/03323

(30) Foreign Application Priority Data
Sep. 1, 2000 (DE) 100435912

(51) Int. Cl. C12Q 1/68

(52) U.S. Cl. 435/6

(57) ABSTRACT

The efficiency of the chemotherapy of malign diseases is limited by resistances vis-à-vis the cytostatics used, which resistances are mediated by a plurality of different mechanisms that proceed at the same time or sequentially. The invention relates to the use of a method of establishing resistance profiles using RNA from tissues or cell lines by way of real-time RT PCR technology (carried out, for example, on the “Light Cycler” of Roche Diagnostics GmbH). The invention allows a quantitative analysis of the expressions of different genes that are associated with the development or the intensification or the reduction of resistances. Based thereon it is, for example, possible to establish individual patient resistance profiles that form the molecular-biological base for the selection of appropriate cytostatics before and also during the particular tumor chemotherapy. The inventive method also allows a prognosis of the chances of success (response) of certain chemotherapeutical regimes.

### SUBJECT OF THE INVENTION

The subject of the invention is a method for establishing chemotherapeutic resistance profiles in human tumor tissues or tumor cell lines, using real time PCR technology (performed, e.g., on the Light Cycler, Roche Diagnostics GmbH). These resistance profiles, individual to patients, are produced on the basis of quantitatively determined expressions of resistance-relevant genes. They can then form the molecular biological rationale for the choice of suitable cytostatics in the respective tumor chemotherapy. Furthermore, the chances of success (response) of given chemotherapeutic regimes can be prognostically assessed.

### SCIENTIFIC BACKGROUND

The efficiency of a chemotherapy of malignant diseases is often limited by resistances to the cytostatics used, mediated by many different mechanisms, occurring in parallel or sequentially.

1. The most important mechanism in this context consists of simultaneous resistance against cytotoxic compounds which are structurally and functionally not used. This phenomenon, known as multidrug resistance (MDR), is caused by the expression of MDR-associated genes. Here the genes which code for the ATP-dependent transmembrane drug efflux pumping (ABC transporters) are in the center of interest. The intracellular concentrations of MDR-associated cytostatics are kept low by overexpression and function of these ABC transporters, and the cell is not, or only a little, affected and is resistant. The following genes coding for ABC transporters belong to these MDR-associated genes: the MDR-1 gene (codes for P-glycoprotein), the genes MRP-1, 2, 3, 4, 5, 6, 7 (code for the multi-drug resistance proteins 1-7), and the gene BCRP/MXR/ABCP (codes for an identical protein; different nomenclature due to simultaneous discovery by 3 different groups). The principal cytostatics spectrum of these transporters includes anthracyclines such as doxorubicin and daunorubicin, Vinca alkaloids such as vincristine and vinblastine, epipodophyllotoxins such as etoposide, taxanes such as taxol, and mitoxantrone, but also the transport of, e.g., nucleosides.

2. A further mechanism associated with MDR consists of the subcellular redistribution of substances, e.g. in nucleocytoplasmic transport. The main constituent of the corresponding cell organelle (vault) is the lung resistance protein/major vault protein LRP/MVP.

3. Further genes causing resistance code for cytoplasmic proteins, which are involved in the metabolism or detoxification of cytotoxics: thus e.g. the enzymes glutathione S-transferase (GST) and aldehyde dehydrogenase (ADH) cause cyclophosphamide resistances by means of intracellular detoxification. Further resistances to cytostatics are mediated by, e.g., dihydrofolate reductase (DHFR; against methotrexate), by thymidylate synthase (against 5-fluorodesoxyuridine) or by tubulin (against Vinca alkaloids and taxol).

4. Nuclear gene products can also cause resistances to cytostatics. Thus, e.g., the enzymes topoisomerase I (resistance against camptothecin) and II (against doxorubicin and etoposide) are involved in the repair of cytostatic-induced DNA damage, as likewise are methyltransferase (MGMT) and methylpurine glycosylase (MPG; both resistance against alkylating agents). The enzyme superoxide dismutase (MnSOD, resistance against anthracyclines) protects from oxidative DNA damage. Also belonging to this group of nuclear gene products causing resistance are the “DNA mismatch repair” genes such as, e.g., MLH1, MSH2 and MSH6, and also PMS1 and PMS2.

5. Furthermore, apoptosis-regulating genes (e.g., Bcl-2, Bax) and also cell cycle involved genes (e.g., p53, MDM2) also belong to those which at least participate in the existence or increase of resistance to cytostatics.

Methods

Standard techniques, such as, e.g., the northern blot methods, can be used for the detection of all of these genes, though no quantitative statements regarding the respective degree of expression can be made by means of such techniques. PCR-based methods such as MIMIC PCR as a very costly and semi-quantitative PCR variant, are not suitable for investigating the expressions of a panel of genes on numerous tissues. The densitometric evaluation of PCR products after gel-electrophoretic separation is likewise found to be difficult. Therefore the method of real time RT PCR is to be used here for the quantification of gene expression, and can be performed, e.g., on the Light Cycler (Roche Diagnostics GmbH).

The methodology of analysis of human tumor material is described hereinafter. Cryosections are prepared for expression analysis from biopsies or resections, directly shock-frozen in the operating theater. Since the methods of microdissection are generally used, the cryosections are examined by a pathologist in order to purposefully microdissect tumor cell populations or normal tissues respectively. This procedure offers the advantage of comparability of the following expression analyses of defined malignant tissues and normal tissues (e.g., both cell areas from the same section). The total cellular RNA is then isolated from these microdissected tissues. Expression analysis is performed on the mRNA level using the Light Cycler system with real time RT PCR and 50 ng of total cellular RNA according to the manufacturer's protocol. The amplification products can be detected either by the intercalation of a fluorescent dyestuff (SYBR Green) or sequence-specifically detected by the use of fluorescent-labeled oligos which hybridize between the primers. Quantification takes place by means of gene-specific transcripts, which are used in parallel in serial dilutions (usually 10⁸, 10⁷, 10⁶, 10⁵). The production of these transcripts took place by means of cloning the corresponding gene-specific cDNA or fragments thereof in special plasmids (e.g., with SP6, T3 or T7 promoters for the corresponding DNA-dependent RNA polymerases). Quality control of the obtained PCR fragments vs. primer dimers takes place by melting point analysis. Visual monitoring can be performed with conventional gel electrophoresis.

For evaluation of the degree of expression of the MDR genes in the tumors, so-called control cell lines are used in parallel. These human cell lines are respectively present as parental lines and also as chemo-resistant variants. Over-expressions, e.g. of specific resistance genes, are characterized on both expression levels: on the RNA level with real time RT PCR, and on the protein level by means of FAC scan analysis with monoclonal antibodies. Furthermore, functional parameters are determined, e.g. in the adriamycin accumulation assay and in the rhodamine influx/efflux assay. These characterizations form the basis for the evaluation of gene expressions in human tissues or cell lines, since in each RT PCR run, RNA of the corresponding cell line pair is used in parallel as a positive control.

The use of the real time RT PCR technology is already described in the oncological literature, e.g. for the quantitative detection of the oncogene MET as a marker for tumor cells in lymph node metastases (G. Cortesina et al., Int. J. Cancer 89:286-292, 2000) or for the detection of a minimal residual disease in breast cancer (M. Giesing et al., Int. J. Biol. Markers 15: 94-99, 2000), in lymphomas (J. G. Sharp et al., Cancer Metastasis Rev. 18: 127-142, 1999), in acute myeloid leukemia (T. Sugimoto et al., Am. J. Hematol. 64: 101-106, 2000) or in chronic myeloid leukemia (M. Emig et al., Clin. Cancer Res. 13: 1825-1832, 1999). This technique has not heretofore been used for the theme of chemotherapy resistance.

### EXAMPLE OF USE

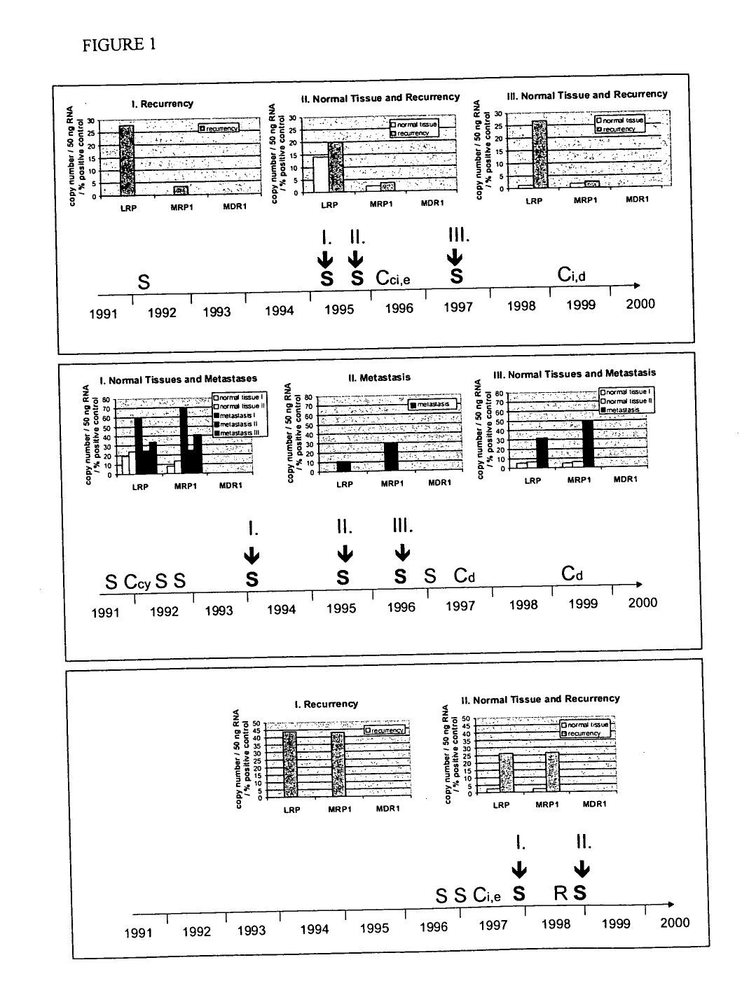

Using the procedure described hereinabove, expression analyses have already been performed of resistance-associated genes in human tumors such as sarcomas and melanomas (MDR1, MRP1, LRP) and also in human tumor cell lines such as colon carcinoma and gastric carcinoma cell lines (MDR1, MRP1, LRP, BCRP). A few examples of the expression of the LRP gene in human sarcomas and the corresponding normal tissues are shown (FIG. 1):

Legend for FIG. 1

Exemplary selected resistance profiles of leiomyosarcoma patients #1-#3, were produced by means of quantitative real time RT PCR. The expression analysis took place on tissues which were obtained at the boldface printed treatment time points. S=surgery, C=chemotherapy, H=hyperthermia, R=radiotherapy, ci=cisplatin, cy=CYVADIC, d=dacarbazine, e=epirubicin, i=iphosphamide, t=TNF, me=meiphalane,

1. Use of a method for determining resistance profiles in tissues or cell lines, wherein it quantitatively determines the RNA expression of defined genes
(a) by means of intercalation of a fluorescent dyestuff (e.g., SYBR Green), or
(b) by means of the use of so-called Taqman probes (labeled at the 5′ and 3′ ends), or
(c) by means of hybridization probes (2 hybridization probes, respectively labeled at the 3′ or 5′ end),
(d) singularly (the expression of one gene is detected in one probe in one run), or
(e) multiplexedly (the expression of several genes is simultaneously detected in one probe in one run).

2. Use according to claim 1, wherein the RNA is isolated from:
(a) human tissues, such as normal tissues or tumor tissues,
(b) from tissues of in vivo models,
(c) from cell lines.

3. Use according to claims 1 and 2, wherein gene-specific primers or primers and probes, are used for the expression analysis of genes which take part in the process of the origination, amplification, and/or reduction of resistances, such as:
(a) genes for transmembrane ABC transporters such as MDR1, MRP1, 2, 3, 4, 5, 6, 7, BCRP/MXR/ABCP,
(b) genes for nucleocytoplasmic transport such as LRP/MVP,
(c) genes for cytoplasmic enzymes such as GST, ADH, DHFR, thymidylate synthase, or tubulin,
(d) genes for nuclear proteins such as topoisomerase I and II, MGMT, MPG, MnSOD, MLH1, MSH2, MSH6, PMS1 and PMS,
(e) genes for apoptosis or time cycle involved proteins such as Bcl-2, Bax, p53, MDM2.

4. Use according to claims 1-3, wherein the expression profile
(a) detects the intrinsic expression status, and/or
(b) detects the expression status after influence due to external factors, such as e.g. in a tumor therapy, and thereby makes possible the determination of potential therapy-conditioned gene modulations.

5. Use according to claims 1-4, wherein
(a) the selection of the cytostatics before a tumor chemotherapy takes place based on the individual, intrinsic resistance profile,
(b) the choice of the cytostatics during a tumor chemotherapy takes place based on the individual, but however modulated resistance profile, and
(c) the chances of success (response) of given chemotherapeutic regimes is assessed.
